(19) United States
(12) Patent Application Publication
(10) Pub. No.: US 2011/0288376 A1
(43) Pub. Date: Nov. 24, 2011

(54) METHOD AND APPARATUS FOR RETRACTION OF PANNUS

(75) Inventor: Joshua A. Tepper, Chicago, IL (US)

(73) Assignee: TZ MEDICAL-TEPPER, LLC, LAKE OSWEGO, OR (US)

(21) Appl. No.: 13/112,205

(22) Filed: May 20, 2011

Related U.S. Application Data
(60) Provisional application No. 61/346,569, filed on May 20, 2010.

(51) Int. Cl. A61B 1/32

(52) U.S. Cl. 600/210

(57) ABSTRACT

A pannus retraction device includes a pannus contacting portion and a femoral region access opening. The device is positioned on the patient's thigh, and is moved to press the pannus away from the femoral access region, and to position the access opening in a desired zone. The device is secured in position so that the pannus is held away during a medical procedure requiring access to the femoral head.

### CROSS-REFERENCE TO RELATED APPLICATIONS

This application is a non-provisional of and claims priority of U.S. provisional patent application 61/346,569, filed May 20, 2010.

### BACKGROUND OF THE INVENTION

This invention relates to access to femoral artery for medical procedures, and more particularly to a method and apparatus for retracting pannus for enabling access to the femoral artery.

The femoral artery is the most commonly used arterial access for coronary, visceral, and peripheral interventions. Pannus (overhanging fat) must be retracted in order to palpate the femoral pulse and safely access the femoral artery. Overlying pannus inhibits the ability to successfully apply manual compression following removal of the arterial access placing patients at increasing risk for peri-procedural complications. Currently there is no reproducible, safe, or easy way to successfully retract pannus.

A typical method currently employed is to roll up a bed sheet, position the central portion of the sheet underneath the pannus, and have 2 assistants holding opposite ends of the sheet, pulling the sheet so as to retract the pannus sufficiently to allow access to the femoral artery. This method has many undesirably limitations, including, for example, the requirement of personnel to hold the sheet ends, resulting fatigue of those persons.

Given the increasing prevalence of obesity and the continued trend towards minimally invasive cardiac, oncologic, and vascular interventions which begin with femoral arterial access, it would be desirable to have a method and apparatus to retract pannus away from the femoral head easing and directing safe arterial access while allowing for manual compression and post-procedural arterial access monitoring in obese patients.

### SUMMARY OF THE INVENTION

In accordance with the invention, methods and apparatus for retracting pannus for access to femoral artery.

Accordingly, it is an object of the present invention to provide an improved apparatus and method for retracting pannus to allow access to the femoral artery for medical procedures. It is a further object of the present invention to provide an improved system for use during medical procedures requiring access to the femoral artery, when the patient is obese.

The subject matter of the present invention is particularly pointed out and distinctly claimed in the concluding portion of this specification. However, both the organization and method of operation, together with further advantages and objects thereof, may best be understood by reference to the following description taken in connection with accompanying drawings wherein like reference characters refer to like elements.

### BRIEF DESCRIPTION OF THE DRAWINGS

FIG. 1 is a perspective view of a patient with a device in accordance with a preferred embodiment of the invention;

FIG. 2 a more detailed view of the contacting portion of the pannus retractor;

FIG. 3 is a top view of the pannus retractor;

FIG. 4 is a top view of an alterative embodiment of a pannus retractor;

FIGS. 5-8 are side views of the pannus retractor of FIG. 4;

FIG. 9 is a front view of the pannus retractor of FIG. 4; and

FIG. 10 is a top view of the pannus retractor of FIG. 4.

### DETAILED DESCRIPTION

The system according to a preferred embodiment of the present invention comprises a portion for pushing the pannus away from the femoral artery area, and an opening positionable over an arterial access zone, for monitoring access to the artery throughout the procedure and during post-procedural recovery.

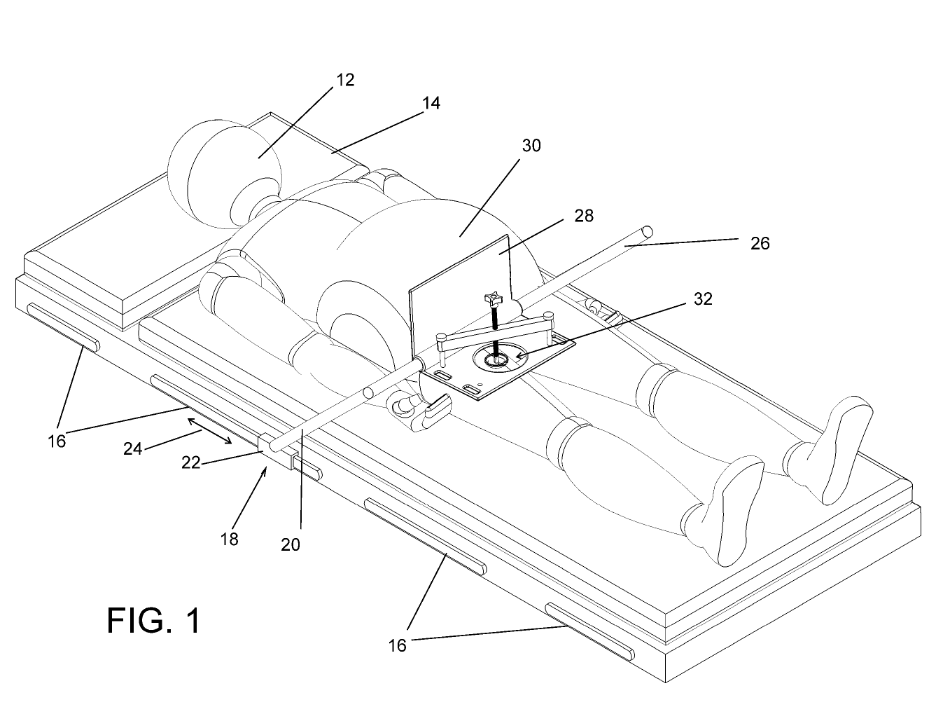

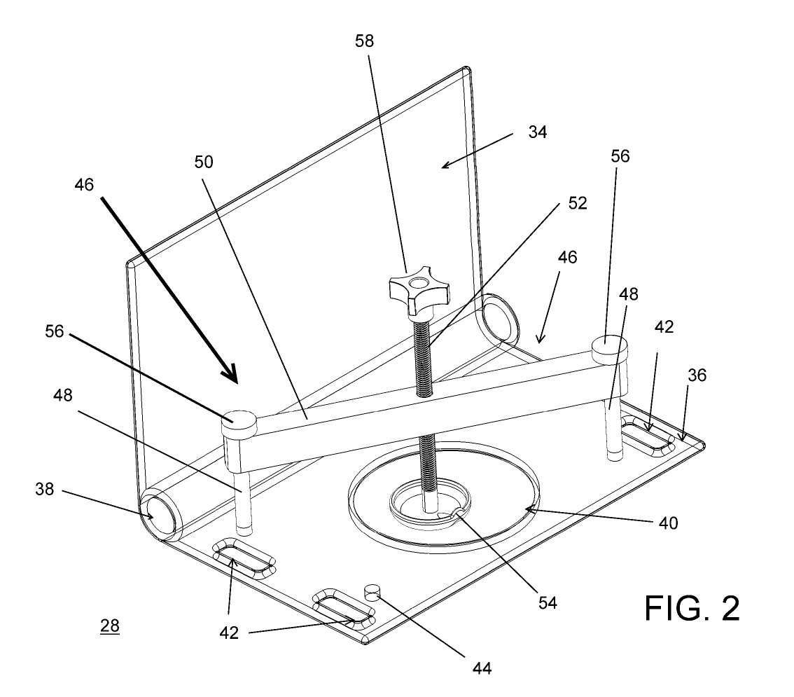

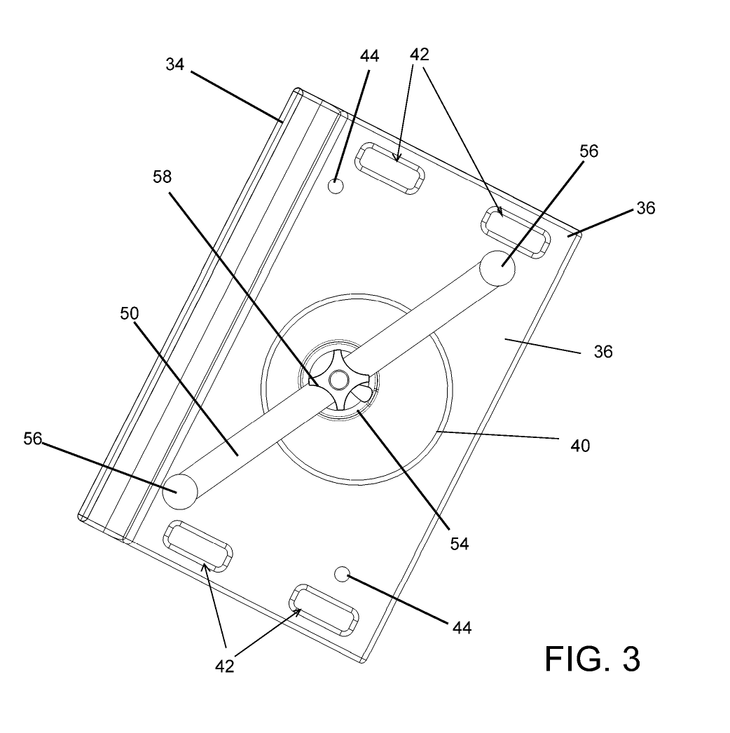

Referring to FIG. 1, a perspective view of a patient with a device in accordance with the invention, a patient 12 is lying on a procedure table 14. The table includes plural attachment bars 16 to enable secure attachment of devices thereto, such as the pannus retractor assembly 18.

The pannus retractor assembly comprises an angledly-upwardly extending bar 20, which is mounted to bar slide 22, adapted for lateral sliding travel in the direction of arrow 24 along a particular attachment bar 16. Bar 20 connects to horizontal bar 26 which extends across the table, above the legs of the patient. Bar 26 passes through a bar receiving portion 38 of a patient contacting part 28, as discussed hereinbelow. In operation, the bar slide is mounted to an appropriate attachment bar 16, and the bar slide is moved toward the patient's head, which moves the patient contacting part 28 in a corresponding direction, resulting in the pannus 30 being pushed away from the femoral access region 32. Once moved a sufficient amount to move the pannus away, the attachment bars are locked in position by means not show in FIG. 1. The retraction and locking may be accomplished by a ratchet mechanism, for example, or other suitable means to provide mechanical advantage for easier retraction as well as locking in place and release for when the procedures are competed.

Referring to FIG. 2, a more detailed view of the contacting portion of the pannus retractor, in conjunction with FIG. 1, the contacting part 28 includes an upwardly extending pannus pressing plate 34 and a horizontal femoral access plate 36, with a bar receiving portion 38 for receiving horizontal bar 26 therethrough, suitably with the opening of portion 38 being such to allow positional movement of the contacting part along the horizontal bar 26 to enable positional placement of the femoral access plate portion in a desired position.

The femoral access plate 36 includes a femoral access opening 40 which provides access to the patient's femoral artery region while the pannus is retracted. Strap receiving openings 42 and tool mount holes 44 are provided on the femoral access plate 36, 4 such openings and mount holes being employed in the illustrated embodiment, positioned approximately adjacent the 4 corners of the plate 36.

In the illustrations, a particular tool 46 is provided, where two threaded mount rods 48 secure a tool mounting bar 50 to the femoral plate. Threaded rods 48 include knobs 56 to allow hand turning thereof for installation and adjustment. Mounting bar 50 supports a tool 52, which in the illustrated case is via threaded engagement to allow vertical positional adjustment of an end effector 54 held at the end of the tool. The end effector 54 can comprise, for example, a femoral arterial flow sensor, such that the flow through the femoral artery can be continuously monitored during procedures to ensure sufficient flow to the patient's extremities is being maintained. A knob 58 at the top of the threaded tool 52 allows adjustment of the tool.

Once the pannus retractor is in desired final position, the pannus retractor is secured in position which may be accomplished by use of a locking member, such as a telescoping rod member that locks into a particular extended length, attached to the procedure table 14 or other anchor point. The telescoping rod can be in either a pulling or pushing configuration, holding the pannus retractor in place by pushing against movement or pulling away from movement. Once the procedures are completed, the rod can be unlocked to allow the pannus retractor to be moved and end the retraction process. Further, either during procedures or after procedures have been completed and the patient is to be moved from the procedure table to a recovery table, the strap receiving openings 42 can be employed to strap the retractor to the patient's leg or other securement points on a bed, to allow the retraction to continue during a post procedure period during which monitoring of the arterial flow may still be desirable, for example, during a recovery time taken to ensure proper clotting at the femoral access opening that was made during the procedure.

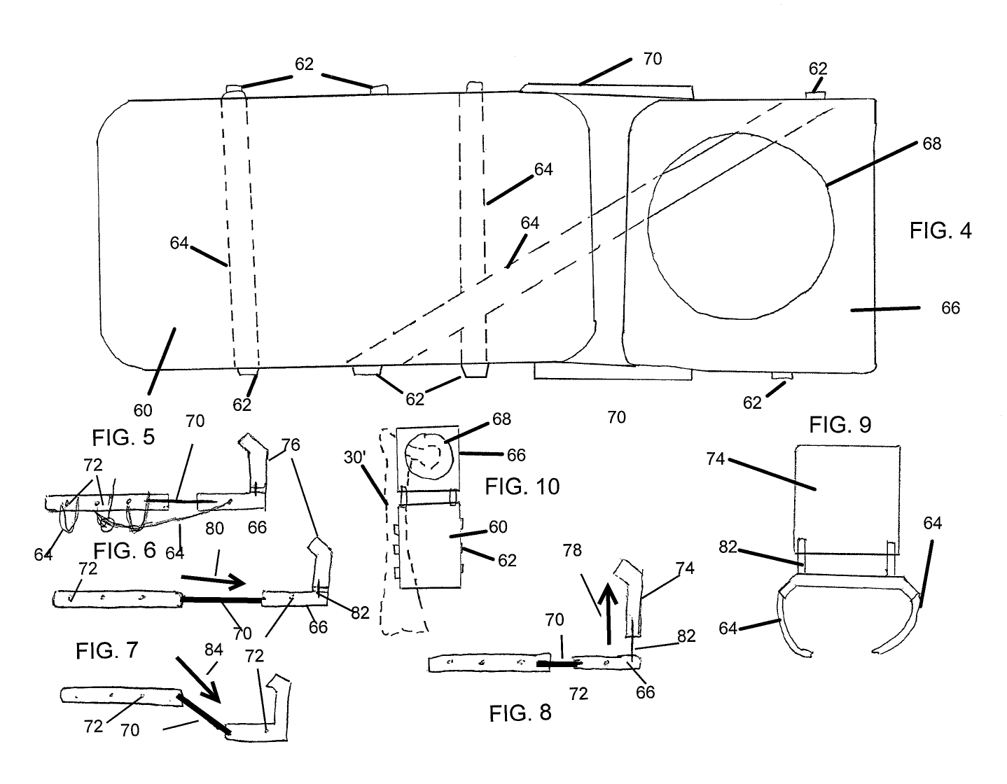

Referring to FIGS. 4-10, an alterative embodiment of the pannus retractor is illustrated. This embodiment employs a substantially rectangular base member 60 adapted for placement on the patient's thigh region, with plural strap attachment points 62 spaced along 2 sides thereof, to receive straps 64 which loop under the patient's leg to allow the base to be strapped to the patient (or alternatively to some fixture points on the table underneath the patient. The base member 60 is suitably convex so as to conform to the patient's thigh. A femoral access plate 66 is provided with an access opening 68 to provide access to the femoral head when the plate is in position. Strap attachment points 62 are also available on plate 66 and straps may be attached to the plate 66 and looped under the patient and attached to the base, for example. Spacer bars 70 are provided on 2 sides of the base/access plate, attaching to both the base and access plate to define the distance 74 between the base and access plate. The access plate may be extended cranially in the direction of arrow 80 (FIG. 6) after the base is strapped to the patient, to move the access plate into position, at which time the spacer bars are put into place to maintain the access plate in position. The spacer bars suitably engage with pins that are received in corresponding openings 72 (visible in FIGS. 5-8) in the sides of the base and access plates.

Referring to FIGS. 5-8, side views of the pannus retractor, a pannus contacting plate 76 attaches to the end of the plate 66 distal from the base 60, and may be extensible in the direction of arrow 78 (vertically) as shown in FIG. 8 to provide different configuration if needed for a particular patient, by use of different lengths of extension bars 82. In FIG. 7, the ability to extend in a direction 84, both cranially and downwardly is illustrated. The device may employ a turnable crank to advance the femoral contacting plate and pannus contacting portion cranially (towards the head) pushing the pannus away from the femoral head. A standard sterile drape with femoral head cutout is placed over the device during procedures. FIGS. 9 and 10 provide front and top views of the pannus retractor.

Accordingly, an improved method and apparatus are provided for pannus retraction to allow access to the femoral head during procedures employing femoral artery catheters, for example. A secure retraction is accomplished without requiring personnel to hold the pannus in a retracted state as is the case with the prior art. While a preferred embodiment of the present invention has been shown and described, it will be apparent to those skilled in the art that many changes and modifications may be made without departing from the invention in its broader aspects. The appended claims are therefore intended to cover all such changes and modifications as fall within the true spirit and scope of the invention.

What is claimed is:

1. A device for retracting pannus on a patient, comprising:
a femoral region contacting plate;
a pannus contacting plate; and
a securement portion for enabling securing the device in a position where pannus is retracted.

2. The device according to claim 1, wherein said securement portion comprises attachment members for enabling attachment of securing straps.

3. The device according to claim 1, wherein said securement portion comprises a positioner receiving portion for receiving a positioning device therein to cause said device to move with movement of the positioner.

4. The device according to claim 1 wherein said femoral region contacting plate comprises a femoral access opening for enabling access to a femoral region of the patient.

5. The device according to claim 1, further comprising tool mount receiving portion for enabling mounting of a tool to said device.

6. A method for retracting pannus on a patient for enabling access to a femoral head region, comprising:
providing a pannus contacting portion for pressing against the pannus;
providing a femoral region access opening;
moving the pannus contacting portion to push the pannus away from the femoral region and to position the femoral region access opening over the femoral head region.

7. The method according to claim 6, further comprising providing attachment members for enabling attachment of securing straps for the pannus contacting portion.

8. The method according to claim 6, further comprising providing a tool mount receiving portion for enabling mounting of a tool to said device.
